(19) United States
(12) Patent Application Publication
(10) Pub. No.: US 2017/0007136 A1
(43) Pub. Date: Jan. 12, 2017

(54) METHODS, SYSTEMS AND NON-TRANSITORY COMPUTER-READABLE RECORDING MEDIA FOR MONITORING BLOOD PRESSURE IN REAL TIME

(71) Applicant: HUINNO, CO., LTD, Seongnam (KR)

(72) Inventor: Yeong Joon GIL, Busan (KR)

(21) Appl. No.: 14/779,851

(22) PCT Filed: Jun. 5, 2015
(86) PCT No.: PCT/KR2015/005680

§ 371 Date: Sep. 28, 2016

(51) Int. Cl. A61B 5/021; A61B 5/145; A61B 5/00; A61B 5/0205; A61B 5/0402; A61B 5/0225

(52) U.S. Cl. CPC A61B 5/02125; A61B 5/0402; A61B 5/02255; A61B 5/0002; A61B 5/0205; A61B 5/14542; A61B 5/7278

(57) ABSTRACT

Methods, systems, and non-transitory computer-readable recording media for monitoring blood pressure are provided. The method includes calculating an estimate of deviations between every two pulse transit time (PTT) values measured at an interval of a first period, and estimating a PTT value to be measured the first period after a current time. The method also includes calculating an estimate of deviations between every two oxygen saturation values measured at an interval of a second period, and estimating an oxygen saturation value to be measured the second period after the current time. The method additionally includes calculating current systolic blood pressure and diastolic blood pressure based on an electrocardiogram (ECG) value measured at the current time, the estimated PTT value, and the estimated oxygen saturation value.

### CROSS-REFERENCE TO RELATED APPLICATIONS

This application is a continuation of International Application No. PCT/KR2015/005680, filed on Jun. 5, 2015, which claims priority to Korean Application No. 10-2015-0012855 filed on Jan. 27, 2015. The applications are incorporated by reference herein.

### TECHNICAL FIELD

The present disclosure relates to methods, systems, and non-transitory computer-readable recording media for monitoring blood pressure in real time.

### BACKGROUND

Due to rapid developments in science and technology, the quality of life of the entire human race has been improved, bringing many changes to the medical environment. Generally, it may take a few hours or days to have medical images, such as X-ray, computerized tomography (CT) or functional magnetic resonance imaging (fMRI) images, interpreted. However, picture archive communication systems (PACS) have been introduced that capture medical images and transmit the captured medical images to the screen of a radiologist's monitor for immediate interpretation by the radiologist. Also, ubiquitous-Healthcare (u-Healthcare)-related medical appliances that enable patients to check their blood sugar and blood pressure anytime anywhere have been widely distributed and are increasingly being used at homes or offices by many diabetic or hypertensive patients.

In particular, in the case of hypertension, which is known to be a major cause of various diseases and has an ever-increasing prevalence, systems are needed for regularly measuring blood pressure and reporting it in real time. Research has been ongoing on such systems. A u-Healthcare technique has been used in which a blood pressure measuring sensor is inserted into the pulmonary artery of a patient with a chronic heart disease to measure the blood pressure of the patient and transmit the result of the measurement to a doctor, in real time, via radio communication. The doctor may then deliver a prescription to the patient while monitoring variations in the pulmonary blood pressure of the patient from a remote place. This u-Healthcare technique allows the number of the patient's visits to the doctor to be considerably reduced. However, the technique requires an invasive measurement process even though it can continuously and precisely measure blood pressure, and the technique may thus face difficulties and risks (such as possible damage or inflammation to the arteries).

Accordingly, systems are needed that are capable of non-invasively measuring blood pressure in real time without the need to insert a blood pressure measuring sensor in the artery. Additionally, ways are needed to allow a user to correct his or her blood pressure through biofeedback of the blood pressure monitored and measured in a ubiquitous environment. A blood pressure measurement method exists in which a cuff is attached to the arm of a user to measure the blood pressure. However, this blood pressure measurement method requires either the user or somebody else (in a case when the user is busy working or resting) to operate a blood pressure measuring device to measure blood pressure. Accordingly, it may be difficult to continuously measure blood pressure. Also, it takes several dozens of seconds to measure blood pressure through this approach.

Thus, in order to alert people to the danger of hypertension and to help them receive timely care in case of an emergency, a technique is needed to continuously measure blood pressure and report blood pressure in real time so as to help people prevent and manage hypertension.

### SUMMARY

The present disclosure relates to at least the problems and disadvantages described above and relates to providing at least the advantages described below.

An exemplary embodiment of the present disclosure provides a method and system for calculating blood pressure without a time delay by calculating an estimate of deviations between every two pulse transit time (PTT) values measured at an interval of a first period, using PTT values measured for a predefined amount of time, estimating a PTT value to be measured the first period after a current time, based on the PTT value deviation estimate and a PTT value measured at the current time. The method and system also may include calculating an estimate of deviations between every two oxygen saturation (SpO₂) values measured at an interval of a second period using oxygen saturation values measured for the predefined amount of time, and estimating an oxygen saturation value to be measured the second period after the current time based on the oxygen saturation value deviation estimate and an oxygen saturation value measured at the current time. The method and system may additionally include calculating current systolic or diastolic blood pressure based on an electrocardiogram (ECG) value measured at the current time, the estimated PTT value and the estimated oxygen saturation value.

In accordance with another exemplary embodiment of the present disclosure, a method of monitoring blood pressure may include calculating an estimate of deviations between every two PTT values measured at an interval of a first period using PTT values measured for a predefined amount of time, and estimating a PTT value to be measured the first period after a current time based on the PTT value deviation estimate and a PTT value measured at the current time. The method may also include calculating an estimate of deviations between every two oxygen saturation values measured at an interval of a second period using oxygen saturation values measured for the predefined amount of time, and estimating an oxygen saturation value to be measured the second period after the current time based on the oxygen saturation value deviation estimate and an oxygen saturation value measured at the current time. The method may additionally include calculating current systolic blood pressure based on an ECG value measured at the current time, the estimated PTT value, and the estimated oxygen saturation value.

In accordance with another exemplary embodiment of the present disclosure, a method of monitoring blood pressure may include calculating an estimate of deviations between every PTT values measured at an interval of a first period using PTT values measured for a predefined amount of time, and estimating a PTT value to be measured the first period after a current time based on the PTT value deviation estimate and a PTT value measured at the current time. The method may also include calculating an estimate of deviations between every two oxygen saturation values measured at an interval of a second period using oxygen saturation values measured for the predefined amount of time, and estimating an oxygen saturation value to be measured the second period after the current time based on the oxygen saturation value deviation estimate and an oxygen saturation value measured at the current time. The method may additionally include calculating current diastolic blood pressure based on the estimated PTT value and the estimated oxygen saturation value.

In accordance with another exemplary embodiment of the present disclosure, a system for monitoring blood pressure may include a deviation estimator configured to calculate an estimate of deviations between every two PTT values measured at an interval of a first period using PTT values measured for a predefined amount of time, and configured to estimate a PTT value to be measured the first period after a current time based on the PTT value deviation estimate and a PTT value measured at the current time. The deviation estimator may also be configured to calculate an estimate of deviations between every two oxygen saturation values measured at an interval of a second period using oxygen saturation values measured for the predefined amount of time, and configured to estimate an oxygen saturation value to be measured the second period after the current time based on the oxygen saturation value deviation estimate and an oxygen saturation value measured at the current time. The system may also include a blood pressure calculator configured to calculate current systolic blood pressure based on an ECG value measured at the current time, the estimated PTT value, and the estimated oxygen saturation value.

In accordance with another exemplary embodiment of the present disclosure, a system for monitoring blood pressure may include a deviation estimator configured to calculate an estimate of deviations between every PTT values measured at an interval of a first period using PTT values measured for a predefined amount of time, and configured to estimate a PTT value to be measured the first period after a current time based on the PTT value deviation estimate and a PTT value measured at the current time. The deviation estimator may also be configured to calculate an estimate of deviations between every two oxygen saturation values measured at an interval of a second period using oxygen saturation values measured for the predefined amount of time, and configured to estimate an oxygen saturation value to be measured the second period after the current time based on the oxygen saturation value deviation estimate and an oxygen saturation value measured at the current time. The system may also include a blood pressure calculator configured to calculate current diastolic blood pressure based on the estimated PTT value and the estimated oxygen saturation value.

In accordance with another exemplary embodiment of the present disclosure, a non-transitory computer-readable recording medium is provided having recorded thereon a computer program for executing any one of the aforementioned methods of monitoring blood pressure. According to the present disclosure, biometric signals such as ECG, photoplethysmogram (PPG), and oxygen saturation signals are measured only for a predefined amount of time only at an initial use. Once the biometric signals are measured, it is possible to continue to precisely calculate blood pressure without a time delay.

The present disclosure is not restricted to those embodiments set forth herein. The above and other embodiments of the present disclosure are exemplary in nature and will become more apparent to one of ordinary skill in the art to which the present disclosure pertains by referencing the detailed description of the present disclosure given below.

Other features and embodiments will be apparent from the following detailed description, the drawings, and the claims.

### BRIEF DESCRIPTION OF THE DRAWINGS

FIG. 1 is a schematic block diagram illustrating an entire system according to an exemplary embodiment of the present disclosure;

FIG. 2 is a block diagram illustrating an apparatus for monitoring blood pressure, according to an exemplary embodiment of the present disclosure; and

FIGS. 3 and 4 are graphs showing PTT values measured for two minutes according to an exemplary embodiment of the present disclosure and deviations among the PTT values.

### DETAILED DESCRIPTION

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Although exemplary embodiments may be described as using a plurality of units to perform the exemplary process, it is understood that the exemplary processes may also be performed by one or plurality of modules. Additionally, it is understood that the term controller/control unit refers to a hardware device that includes a memory and a processor. The memory is configured to store the modules and the processor is specifically configured to execute said modules to perform one or more processes which are described further below.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Embodiments of the present disclosure are described in detail below with reference to the accompanying drawings. The embodiments of the present disclosure are sufficiently described in detail such that those skilled in the art may carry out the present disclosure. It should be understood that although various embodiments of the present disclosure are different from each other, they need not be mutually exclusive. For example, in regard to an embodiment, specific forms, structures, and characteristics described herein can be realized through another embodiment without departing from the spirit and scope of the present disclosure. Moreover, it should be understood that locations or arrangements of separate elements within the disclosed embodiments can be changed without departing from the spirit and scope of the present disclosure. Accordingly, detailed descriptions which will be given below are not intended to be restrictive, and the scope of the present disclosure, if properly described, should be limited only by the accompanying claims and equivalents thereof. Similar reference numerals shown in the drawings denote elements performing identical or similar functions in several aspects.

Embodiments will hereinafter be described with reference to the accompanying drawings.

### Structure of Entire System

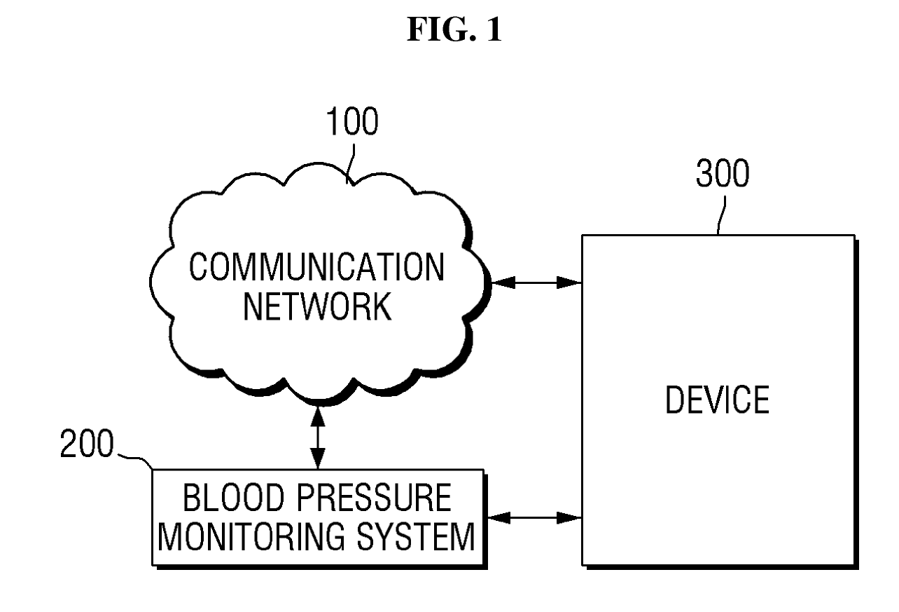

A system for monitoring blood pressure, according to an exemplary embodiment of the present disclosure will hereinafter be described. FIG. 1 is a schematic block diagram illustrating an entire system according to an exemplary embodiment. Referring to FIG. 1, the system may include a communication network 100, a system 200 (hereinafter, the blood pressure monitoring system 200) for monitoring blood pressure, and a device 300.

The communication network 100 may not necessarily be limited to a particular communication method such as a wired or wireless communication method, and may be implemented as various communication networks such as a local area network (LAN), a metropolitan area network (MAN) or a wide area network (WAN). Examples of the communication network 100 may include LANs such as a Wireless-Fidelity (Wi-Fi) network, a Wi-Fi Direct network, a Long-Term Evolution (LTE) Direct network, and a Bluetooth network that are already well known, but the present disclosure is not limited thereto. That is, the communication network 100 may at least partially include a typical wired/wireless communication network, a typical telephone network or a typical wired/wireless television communication network.

The blood pressure monitoring system 200 may calculate blood pressure without a time delay by calculating an estimate of deviations between every two PTT values measured at an interval of a first period using PTT values measured for a predefined amount of time, and configured to estimate a PTT value to be measured the first period after a current time based on the PTT value deviation estimate and a PTT value measured at the current time. The system 200 may be configured to then calculate an estimate of deviations between every two oxygen saturation values measured at an interval of a second period using oxygen saturation values measured for the predefined amount of time, and configured to then estimate an oxygen saturation value to be measured the second period after the current time based on the oxygen saturation value deviation estimate and an oxygen saturation value measured at the current time. The system 200 may then be configured to calculate current systolic or diastolic blood pressure based on an ECG value measured at the current time, the estimated PTT value, and the estimated oxygen saturation value.

The functions of the blood pressure monitoring system 200 will be described later in further detail. The blood pressure monitoring system 200 has been described above, but the present disclosure is not limited thereto. That is, some of the functions or elements of the blood pressure monitoring system 200 may be implemented or incorporated into the device 300, if necessary.

The device 300 may be a digital device capable of accessing and communicating with the blood pressure monitoring system 200, and any digital device equipped with memory and a microprocessor and having computational capabilities may be used as the device 300. The device 300 may be a wearable device such as smart glasses, a smart watch, a smart band, a smart ring, and/or a smart necklace, or a device such as a smartphone, a smart pad, a desktop computer, a notebook computer, a workstation, a personal digital assistant (PDA), a web pad, and/or a mobile phone. The device 300 may measure a biometric signal for monitoring a blood pressure from the human body or may provide blood pressure monitoring information to a user.

The device 300 may also include application programs for performing predetermined functions. The application programs may exist in the device 300 in the form of programs. The programs may be similar to a deviation estimator 210, a blood pressure calculator 220, a communicator 230 and a controller 240 of the blood pressure monitoring system 200. The application programs may be replaced with hardware or firmware devices capable of performing the same functions as, or at least equivalent functions to, the application programs, if necessary. Communicators, memory, programs, calculators, estimators, controllers, modules, and/or any units may be operated by a master controller having a memory and a processor.

### Structure of Blood Pressure Monitoring System

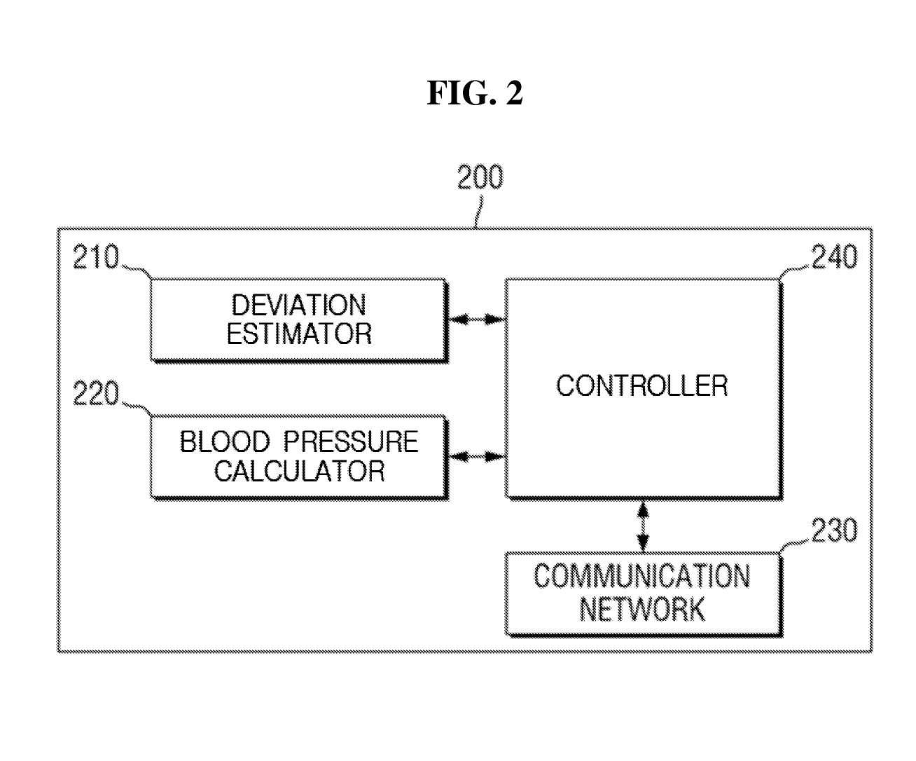

The structure and functions of the blood pressure monitoring system 200 will hereinafter be described. FIG. 2 is a block diagram illustrating an apparatus for monitoring blood pressure according to an exemplary embodiment.

Referring to FIG. 2, the blood pressure monitoring system 200 may include a deviation estimator 210, a blood pressure calculator 220, a communicator 230, and a controller 240. At least some of the deviation estimator 210, the blood pressure calculator 220, the communicator 230, and the controller 240 may be programs communicating with an external system (not illustrated). The programs may be included in the blood pressure monitoring system 200 in the form of operating systems, application programs, or other programs, and may be physically stored in a memory device that is already known in the art. The programs may also be stored in a remote memory device capable of communicating with the blood pressure monitoring system 200. The programs may include routines, sub-routines, programs, objects, components, and data structures that perform particular tasks or particular abstract data types, but the present disclosure is not limited thereto.

The deviation estimator 210 may be configured to calculate an estimate of deviations between every two PTT values measured at an interval of a first period using PTT values measured for a predefined amount of time, and may be configured to estimate a PTT value to be measured the first period after a current time based on the PTT value deviation estimate and a PTT value measured at the current time. Also, the deviation estimator 210 may be configured to calculate an estimate of deviations between every two oxygen saturation values measured at an interval of a second period using oxygen saturation values measured for the predefined amount of time, and may be configured to estimate an oxygen saturation value to be measured the second period after the current time based on the oxygen saturation value deviation estimate and an oxygen saturation value measured at the current time.

More specifically, the predefined amount of time, which is a period for securing sufficient PTT and oxygen saturation data at an initial use, may be set to be longer than the first period or the second period. For example, in response to first and second periods for calculating systolic blood pressure being about 8 seconds and about 42 seconds, respectively, the predefined amount of time for measuring PTT values and oxygen saturation values at an initial use may be set to be longer than about 42 seconds. The PTT value to be measured the first period after the current time may be estimated by adding the PTT value deviation estimate to the PTT value measured at the current time. Similarly, the oxygen saturation value to be measured the second period after the current time may be estimated by adding the oxygen saturation value deviation estimate to the oxygen saturation value measured at the current time.

The PTT value deviation estimate may be an average of the deviations between every two PTT values measured at the interval of the first period. Similarly, the oxygen saturation value deviation estimate may be an average of the deviations between every two oxygen saturation values measured at the interval of the second period.

The deviation estimator 210 may be configured to calculate the PTT value deviation estimate or the oxygen saturation value deviation estimate based on all the deviations between every two PTT values measured at the interval of the first period or all the deviations between every two oxygen saturation values measured at the interval of the second period except for outliers. For example, the deviation estimator 210 may be configured to calculate the PTT value deviation estimate based on all the deviations between every two PTT values measured at the interval of the first period, except for those that fall in about the top 10% range and those that fall in about the bottom 10% range, i.e., based on the PTT value deviations that fall in about the middle 80% range. For this, the deviation estimator 210 may arrange the PTT value deviations in a predetermined order, for example, in ascending or descending order.

The blood pressure estimator 220 may be configured to calculate the current systolic blood pressure based on an ECG value measured at the current time, the estimated PTT value, and the estimated oxygen saturation value. The blood pressure estimator 220 may calculate the current diastolic blood pressure based on the estimated PTT value and the estimated oxygen saturation value.

The calculation of systolic or diastolic blood pressure will hereinafter be described. Systolic blood pressure and diastolic blood pressure may be calculated based on an ECG value, a PTT value, and an oxygen saturation value, as indicated by Equations (1) and (2):

SBP(t)=(a×ECG₍ₜ₊₂₄ₛ₎)+(b×1/(PTT₍ₜ₊₃₂ₛ))+(c×1/(SpO₂₍ₜ₊₇₆ₛ₎))+d (1);

and

DBP(t)=(e×1/(PTT₍ₜ₊₇₀ₛ))+(f×1/(SpO₂₍ₜ₊₁₀₀ₛ)))+g (2).

Equations (1) and (2) may be simplified through time shift into Equations (3) and (4):

SBP(t′)=(a×ECG_{(t′)})+(b×1/(PTT_{t′+8s}))+(c×1/(SpO_{2(t′+42s}))+d (3);

and

DBP(t′)=(e×1/(PTT_{(t′+46s}))+(f×1/(SpO_{2(t′+76s}))+g (4).

In Equations (1) through (4), SBP(t) and DBP(t) denote systolic blood pressure and current diastolic blood pressure, respectively, ECG(t), PTT(t) and SpO₂(t) denote an ECG value, a PTT value, and an oxygen saturation value, respectively, a, b, c, d, e, f and g denote constants (that may vary depending on the environment in which, or the target from which, blood pressure is measured). For example, the constants a, b, c, d, e, f, and g may be 0.115, 4.132, 509.819, −454.111, −3.015, −304.556, and 410.62, respectively.

Referring to Equation (3), to calculate current systolic blood pressure, not only an ECG value ECG_{(t′)} measured at the current time, but also a PTT value PTT_{(t′+8s)} measured about 8 seconds after the current time and an oxygen saturation value SPO_{2(t′+42s)} measured about 42 seconds after the current time, are needed, and thus, it inevitably takes at least about 42 seconds to calculate the current systolic blood pressure. Similarly, referring to Equation (4), to calculate current diastolic blood pressure, a PTT value PTT_{(t′+46s)} measured about 46 seconds after the current time and an oxygen saturation value SPO_{2(t′+76s)} measured about 76 seconds after the current time, are needed, and thus, it inevitably takes at least about 76 seconds to calculate the current diastolic blood pressure.

On the other hand, according to the present exemplary embodiment, a PTT value and an oxygen saturation value to be measured a predetermined amount of time after the current time may be estimated, as discussed above with regard to the deviation estimator 210 and the blood pressure calculator 220. Accordingly, the current systolic blood pressure and the current diastolic blood pressure can be calculated without a time delay of several seconds.

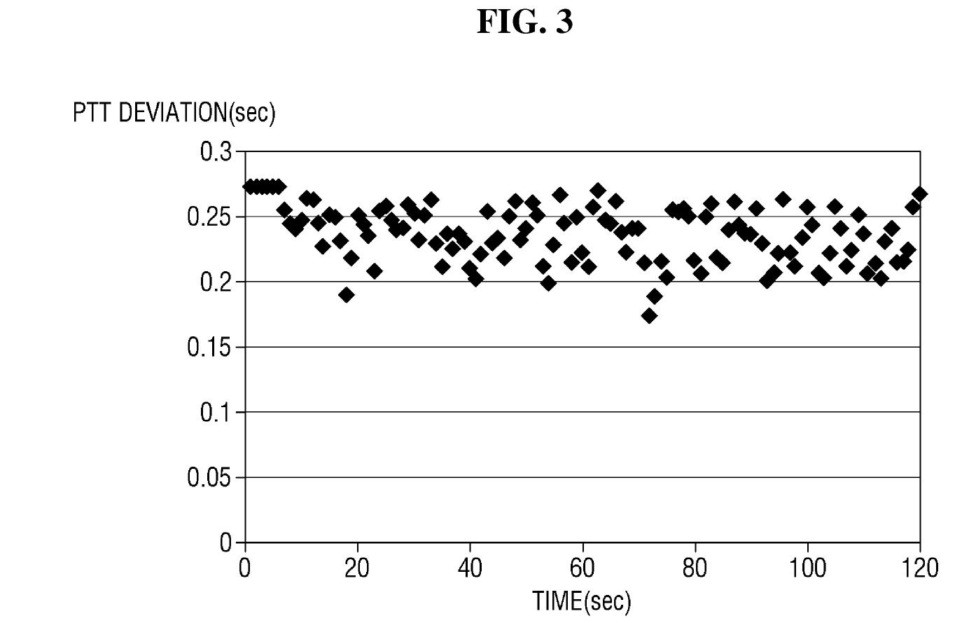

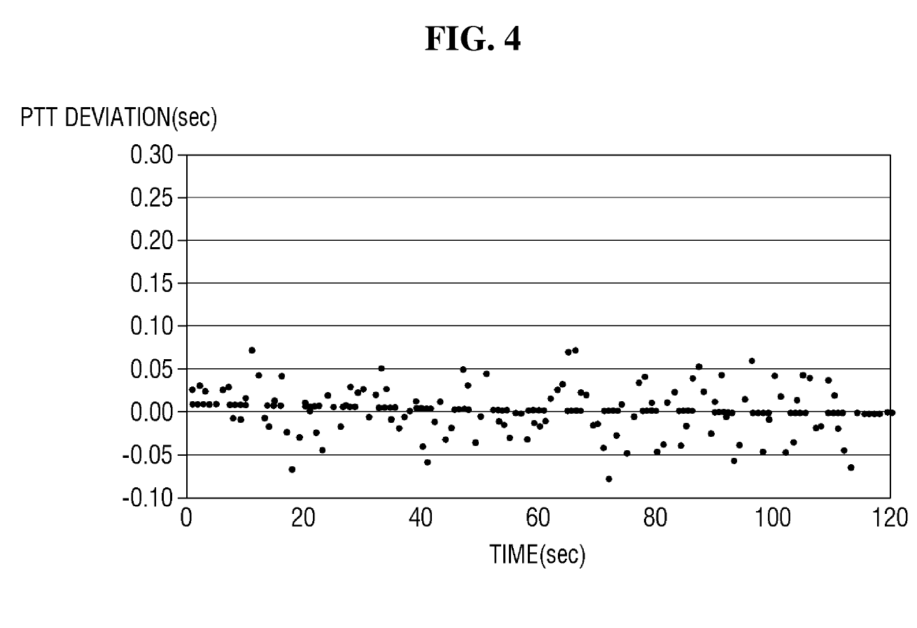

FIGS. 3 and 4 are graphs showing PTT values measured for two minutes according to an exemplary embodiment and deviations among the PTT values. More specifically, FIG. 3 show PTT values measured from a first test subject for two minutes (or 120 seconds). Referring to FIG. 3, the PTT values measured from the first test subject generally range from 200 msec to 300 msec.

FIG. 4 show deviations between every two PTT values (i.e., PTT_{(t′)}-PTT_{(t′+8})) measured 8 seconds apart. Referring to FIG. 4, the average of the PTT value deviations may be almost zero, and particularly, about 0.00214 seconds. Although not specifically illustrated, deviations between every two oxygen saturation values measured at an interval of a predetermined amount of time may be estimated using almost the same method as that of FIGS. 3 and 4. More specifically, test results show that the average of deviations (i.e., SpO_{2(t′})-SpO_{2(t′+8})) between every two oxygen saturation values measured 42 seconds apart is 0.17%.

Equation (3), which is for calculating systolic blood pressure using biometric signals that are actually measured, may be transformed into Equation (5), which is for calculating systolic blood pressure using biometric signals measured based on average deviations, and Equation (5) is as follows:

SBP(t′)=(a×ECG_{(t′)})+(b×1/(PTT_{(t′)}−0.00214))+(c×1/(SpO_{2(t′)}−0.0017))+d (5).

In the present exemplary embodiment, test results show that systolic blood pressure calculated by Equation (3), which uses biometric signals that are actually measured with time delays, is 127.1053 mmHg, and that systolic blood pressure calculated by Equation (5), which uses biometric signals that are estimated based on average deviations, is 125.7324 mmHg. That is, the systolic blood pressure calculated by Equation (5), which uses estimated biometric signals according to the present exemplary embodiment, and the systolic blood pressure calculated by Equation (3), which uses measured biometric signals, is only about 1%. Even though not specifically mentioned herein, tests were conducted on fifty other test subjects than the first test subject, and the results confirm that the error rate between systolic blood pressure calculated on estimated biometric signals according to the present exemplary embodiment and systolic blood pressure calculated based on measured biometric signals is an average of about 1%.

According to the present exemplary embodiment, current systolic blood pressure may be precisely calculated without a time delay. The calculation of systolic blood pressure has been described above, but it is obvious that the present disclosure can also be directly applied to the calculation of diastolic blood pressure. The communicator 230 may allow the blood pressure monitoring system 200 to communicate with an external device.

The controller 240 controls the flow of data among the deviation estimator 210, the blood pressure calculator 220, and the communicator 230. That is, the controller 240 may control the flow of data from an external device or the flow of data between the elements of the blood pressure monitoring system 200, and may thus control the deviation estimator 210, the blood pressure calculator 220, and the communicator 230 to perform their respective functions.

Embodiments of the present disclosure may be implemented in the form of program instructions that can be performed by various computing devices to be thereby recorded in a non-transitory computer-readable recording medium. The non-transitory computer-readable recording medium may include program instructions, data files, and data structures alone or a combination thereof. The program instructions recorded in the recording medium may be specially designed and configured for the present disclosure, or be something well-known to those skilled in the field of computer software. The non-transitory computer-readable recording medium may include magnetic media such as hard disks, floppy disks and magnetic tapes, optical media such as a Compact Disc Read-Only Memory (CD-ROM) and a Digital Versatile Disc (DVD), magneto-optical media such as floptical disks, and hardware devices such as a Read-Only Memory (ROM), a Random Access Memory (RAM) and a flash memory, which are specially configured to store and perform program instructions. Further, the program instructions include a machine language code generated by a compiler and a high-level language code executable by a computer through an interpreter and the like. The hardware devices may be configured to operate as one or more software modules to perform the operations of the present disclosure, and vice versa.

As described above, although the present disclosure has described specific matters such as concrete components, the embodiments and the drawings are provided merely to assist in a general understanding of the present disclosure, and the present disclosure is not limited to the embodiments. Various modifications and changes can be made from the description by those skilled in the art.

Accordingly, the spirit and scope of the present disclosure should not be limited or determined by the above-described embodiments, and it should be noted that not only the claims which will be described below but also their equivalents fall within the spirit and scope of the present disclosure.

1. A method of monitoring blood pressure, comprising:
calculating an estimate of deviations between every two pulse transit time (PTT) values measured at an interval of a first period using PTT values measured for a predefined amount of time, and estimating a PTT value to be measured the first period after a current time based on the PTT value deviation estimate and a PTT value measured at the current time;
calculating an estimate of deviations between every two oxygen saturation values measured at an interval of a second period using oxygen saturation values measured for the predefined amount of time, and estimating an oxygen saturation value to be measured the second period after the current time based on the oxygen saturation value deviation estimate and an oxygen saturation value measured at the current time; and
calculating current systolic blood pressure based on an electrocardiogram (ECG) value measured at the current time, the estimated PTT value, and the estimated oxygen saturation value.

2. The method of claim 1, wherein the predefined amount of time is set to be longer than whichever of the first and second periods is longer than the other.

3. The method of claim 1, wherein the PTT value to be measured the first period after the current time is estimated by adding the PTT value deviation estimate to the PTT value measured at the current time, and
wherein the oxygen saturation value to be measured the second period after the current time is estimated by adding the oxygen saturation value deviation estimate to the oxygen saturation value measured at the current time.

4. The method of claim 1, wherein the PTT value deviation estimate is an average of the deviations between the every two PTT values measured at the interval of the first period, and
wherein the oxygen saturation value deviation estimate is an average of the deviations between the every two oxygen saturation values measured at the interval of the second period.

5. The method of claim 1, wherein the PTT value deviation estimate is calculated based on all the deviations between the every two PTT values measured at the interval of the first period, except for outliers that fall outside a predefined range, and
wherein the oxygen saturation value deviation estimate is calculated based on all the deviations between the every two oxygen saturation values measured at the interval of the second period, except for outliers that fall outside a predefined range.

6. The method of claim 1, wherein the first period is set to be shorter than the second period.

7. The method of claim 1, wherein calculating the current systolic blood pressure includes calculating the current systolic blood pressure in real time without waiting for PTT values and oxygen saturation values to be measured for the predefined amount of time.

8. A method of monitoring blood pressure, comprising:
calculating an estimate of deviations between every two pulse transit time (PTT) values measured at an interval of a first period using PTT values measured for a predefined amount of time, and estimating a PTT value to be measured the first period after a current time based on the PTT value deviation estimate and a PTT value measured at the current time;
calculating an estimate of deviations between every two oxygen saturation values measured at an interval of a second period using oxygen saturation values measured for the predefined amount of time, and estimating an oxygen saturation value to be measured the second period after the current time based on the oxygen saturation value deviation estimate and an oxygen saturation value measured at the current time; and
calculating current diastolic blood pressure based on the estimated PTT value and the estimated oxygen saturation value.

9. A system for monitoring blood pressure, comprising:
a deviation estimator configured:
to calculate an estimate of deviations between every two pulse transit time (PTT) values measured at an interval of a first period using PTT values measured for a predefined amount of time, and to estimate a PTT value to be measured the first period after a current time, based on the PTT value deviation estimate and a PTT value measured at the current time, and
to calculate an estimate of deviations between every two oxygen saturation values measured at an interval of a second period using oxygen saturation values measured for the predefined amount of time, and to estimate an oxygen saturation value to be measured the second period after the current time based on the oxygen saturation value deviation estimate and an oxygen saturation value measured at the current time; and
a blood pressure calculator configured to calculate current systolic blood pressure based on an ECG value measured at the current time, the estimated PTT value, and the estimated oxygen saturation value.

10. A system for monitoring blood pressure, comprising:
a deviation estimator configured:
to calculate an estimate of deviations between every pulse transit time (PTT) values measured at an interval of a first period using PTT values measured for a predefined amount of time, and to estimate a PTT value to be measured the first period after a current time based on the PTT value deviation estimate and a PTT value measured at the current time, and
to calculate an estimate of deviations between every two oxygen saturation values measured at an interval of a second period using oxygen saturation values measured for the predefined amount of time, and to estimate an oxygen saturation value to be measured the second period after the current time based on the oxygen saturation value deviation estimate and an oxygen saturation value measured at the current time; and
a blood pressure calculator configured to calculate current diastolic blood pressure based on the estimated PTT value and the estimated oxygen saturation value.

11. The system of claim 9, wherein the predefined amount of time is set to be longer than whichever of the first and second periods is longer than the other.

12. The system of claim 9, wherein the PTT value to be measured the first period after the current time is estimated by adding the PTT value deviation estimate to the PTT value measured at the current time, and
wherein the oxygen saturation value to be measured the second period after the current time is estimated by adding the oxygen saturation value deviation estimate to the oxygen saturation value measured at the current time.

13. The system of claim 9, wherein the PTT value deviation estimate is an average of the deviations between the every two PTT values measured at the interval of the first period, and
wherein the oxygen saturation value deviation estimate is an average of the deviations between the every two oxygen saturation values measured at the interval of the second period.

14. The system of claim 9, wherein the PTT value deviation estimate is calculated based on all the deviations between the every two PTT values measured at the interval of the first period, except for outliers that fall outside a predefined range, and
wherein the oxygen saturation value deviation estimate is calculated based on all the deviations between the every two oxygen saturation values measured at the interval of the second period, except for outliers that fall outside a predefined range.

15. The system of claim 9, wherein the first period is set to be shorter than the second period.

16. The system of claim 9, wherein calculating the current systolic blood pressure by the blood pressure calculator includes calculating the current systolic blood pressure in real time without waiting for PTT values and oxygen saturation values to be measured for the predefined amount of time.

17. A non-transitory computer-readable recording medium storing a computer program for executing the method according to claim 1.

18. A non-transitory computer-readable recording medium storing a computer program for executing the method according to claim 8.
